(19) United States
(12) Patent Application Publication
(10) Pub. No.: US 2019/0030061 A1
(43) Pub. Date: Jan. 31, 2019

(54) COBALAMIN COMPOSITIONS AND USE THEREOF FOR IMPROVING COGNITIVE FUNCTION

(71) Applicant: NOVA SOUTHEASTERN UNIVERSITY, Fort Lauderdale, FL (US)

(72) Inventors: Mutasem Rawas-Qalaji, Fort Lauderdale, FL (US); Richard Deth, Fort Lauderdale, FL (US)

(73) Assignee: NOVA SOUTHEASTERN UNIVERSITY, Fort Lauderdale, FL (US)

(21) Appl. No.: 16/081,111

(22) PCT Filed: Mar. 1, 2017
(86) PCT No.: PCT/US17/20125

§ 371 Date: Aug. 30, 2018

Related U.S. Application Data
(60) Provisional application No. 62/301,876, filed on Mar. 1, 2016.

(51) Int. Cl. A61K 31/714; A61K 33/00; A61K 31/519; A61K 31/122; A61K 9/00; A23L 33/15; A23L 33/16

(52) U.S. Cl. CPC A61K 31/714; A61K 33/00; A61K 31/519; A23V 2002/00; A61K 9/006; A23L 33/15; A23L 33/16; A61K 31/122

(57) ABSTRACT

The invention provides cobalamin (vitamin B₁₂) nutraceutical compositions and methods for use thereof for improving cognitive function in subjects having disorders of cognitive function such as neurodegenerative disorders, neurodevelopmental disorders, and neuropsychiatric disorders. This improvement in cognitive function is achieved through the ability of the cobalamin (vitamin B₁₂) nutraceutical compositions to increase methylation capacity and vitamin B₁₂ activity in the brains of the subjects.

### FIELD OF THE INVENTION

The invention generally relates to methods and compositions for improving cognitive function; particularly to methods and nutraceutical/pharmaceutical formulations containing cobalamin (vitamin B₁₂) for improving cognitive function; and mostly particularly to methods and nutraceutical/pharmaceutical formulations containing cobalamin (vitamin B₁₂) for increasing methylation capacity in the brain to achieve an improvement in cognitive function.

### BACKGROUND

Metabolically active forms of vitamin B₁₂, methylcobalamin (MeCbl) and adenosylcobalamin (AdoCbl), serve as essential cofactors for two reactions: MeCbl for folate-dependent methylation of HCY to methionine by methionine synthase (MS) in the cytoplasm, and AdoCbl for conversion of methylmalonylCoA to succinylCoA by methylmalonyl CoA mutase in mitochondria (Guéant J L, Caillerez-Fofou M, Battaglia-Hsu S, Alberto J M, Freund J N, Dulluc I et al. Molecular and cellular effects of vitamin B₁₂ in brain, myocardium and liver through its role as co-factor of methionine synthase. Biochimie. 2013; 95:1033-1040 and Gherasim C, Lofgren M, Banerjee R. Navigating the B(12) road: assimilation, delivery, and disorders of cobalamin. J Biol Chem. 2013; 288: 13186-13193). Since MS activity determines the ratio of the methyl donor S-adenosylmethionine (SAM) to the endogenous methylation inhibitor S-adenosylhomocysteine (SAH), MeCbl is poised to influence hundreds of SAM-dependent methylation reactions, affecting nearly every aspect of metabolism. Important among these reactions is methylation of DNA and histones, which combine to exert dynamic epigenetic control over gene expression (Park L K, Friso S, Choi S W. Nutritional influences on epigenetics and age-related disease. Proc Nutr Soc. 2012; 71:75-83). MeCbl is also required for dopamine-stimulated phospholipid methylation, a unique activity of D4 dopamine receptors (Sharma A, Kramer M L, Wick P F, Liu D, Chari S, Shim S. et al. D4 dopamine receptor-mediated phospholipid methylation and its implications for mental illnesses such as schizophrenia. Mol Psychiatry. 1999; 4:235-246) which depends upon MS activity (Waly M, Olteanu H, Banerjee R, Choi S W, Mason J B, Parker B S, et al. Activation of methionine synthase by insulin-like growth factor-1 and dopamine: a target for neurodevelopmental toxins and thimerosal. Mol Psychiatry. 2004; 9:358-370) and has been proposed to play an important role in neuronal synchronization and attention (Kuznetsova A Y, Deth R C. A model for modulation of neuronal synchronization by D4 dopamine receptor-mediated phospholipid methylation. J Comput Neurosci. 2008; 24:314-329). Genetic variants of the D4 receptor have been linked to attention-deficit hyperactivity disorder (ADHD) (Swanson J M, Kinsbourne M, Nigg J, Lanphear B, Stefanatos G A, Volkow N et al. Etiologic subtypes of attention-deficit/hyperactivity disorder: brain imaging, molecular genetic and environmental factors and the dopamine hypothesis. Neuropsychol Rev. 2007; 17: 39-59 and Tarazi F I, Baldessarini R J. Dopamine D4 receptors: significance for molecular psychiatry at the millennium. Mol Psychiatry 1999; 4:529-538), schizophrenia risk (Lai J H, Zhu Y S, Huo Z H, Sun R F, Yu B, Wang Y P, et al. Association study of polymorphisms in the promoter region of DRD4 with schizophrenia, depression, and heroin addiction. Brain Res 2010; 1359:227-232. Cheng J, Wang Y, Zhou K, Wang L, Li J, Zhuang Q, et al. Male-specific association between dopamine receptor D4 gene methylation and schizophrenia. PLoS One. 2014; 9:e89128), and drug addiction (Lai J H, Zhu Y S, Huo Z H, Sun R F, Yu B, Wang Y P, et al. Association study of polymorphisms in the promoter region of DRD4 with schizophrenia, depression, and heroin addiction. Brain Res 2010; 1359:227-232) as well as to human longevity (Grady D L, Thanos P K, Corrada M M, Barnett J C Jr, Ciobanu V, Shustarovich D. et al. DRD4 genotype predicts longevity in mouse and human. J Neurosci. 2013; 33:286-291).

Vitamin B₁₂ is only synthesized by certain bacteria and humans obtain it from animal source foods such as meat, dairy, eggs, and fish (Gille D, Schmid A. Vitamin B12 in meat and dairy products. Nutr Rev. 2015; 73:106-115). A series of chaperones, transport proteins and their receptors (e.g. haptocorrin, intrinsic factor, cubilin, amnionless and megalin) protect vitamin B₁₂ and facilitate its GI absorption and renal reabsorption for its retention. In the general circulation vitamin B₁₂ primarily exists bound to transcobalamin (TC) (Kozyraki R, Cases O. Vitamin B12 absorption: mammalian physiology and acquired and inherited disorders. Biochimie. 2013; 95:1002-1007). Cell surface receptors (TC receptor and/or megalin) bring the Cbl_TC complex into lysosomes where Cbl is processed by MMACHC (methylmalonic aciduria type C and homocystinuria, also known as CblC). MMACHC carries out dealkylation of alkylCbls and decyanation of cyanocobalamin (CNCbl) in glutathione (GSH)-dependent and NADPH-dependent reactions, respectively (Gherasim C, Lofgren M, Banerjee R. Navigating the B(12) road: assimilation, delivery, and disorders of cobalamin. J Biol Chem. 2013; 288: 13186-13193). Formation of active cofactors MeCbl and AdoCbl is then carried out by MMACHC in conjunction with MMADHC (methylmalonic aciduria type D and homocystinuria, also known as CblD) in the cytoplasm and mitochondria, respectively.

The brain exists within a distinct compartment and levels of metabolic resources, including vitamin B₁₂, are reflective of their transport into and out of cerebral spinal fluid (CSF) across the neuroepithelial barrier in the choroid plexus. While factors responsible for vitamin B₁₂ entry into brain have not been fully elucidated, cubilin and megalin, which combine to participate in transport of vitamin B₁₂ in other tissues, are expressed in the choroid plexus (Carro E, Spuch C, Trejo J L, Antequera D, Torres-Aleman I. Choroid plexus megalin is involved in neuroprotection by serum insulin-like growth factor I. J Neurosci. 2005; 25:10884-10893 and Christensen E I, Birn H. Megalin and cubilin: multifunctional endocytic receptors. Nat Rev Mol Cell Biol 2002; 3:256-266) and a role for amnionless has been postulated based upon disturbed vitamin B₁₂ transport into the brain in a patient with a mutation causing Imerslund-Grasbeck syndrome (Luder A S, Tanner S M, de la Chapelle A, Walter J H. Amnionless (AMN) mutations in Imerslund-Grasbeck syndrome may be associated with disturbed vitamin B(12) transport into the CNS. J Inherit Metab Dis. 2008; 31 Suppl 3:493-6). While diet or genetic defects in transport/processing can affect systemic vitamin B12 availability (Stabler SP. Clinical practice. Vitamin B12 deficiency. N Engl J Med. 2013; 368:149-160 and Kirsch S H, Herrmann W, Obeid R. Genetic defects in folate and cobalamin pathways affecting the brain. Clin Chem Lab Med. 2013; 51:139-155), there have been relatively few direct studies of vitamin B₁₂ status in human brain (Worm-Peterson J. Vitamin B12 haemoglobin and iron concentration in human brain tissue. Acta Neurol Scand. 1964; 40: 241-8 and Baker H, Frank O, Chen T, Feingold S, DeAngelis B, Baker E. Vitamin content of some normal human brain segments. J Neurosci Res. 1984; 11: 419-35) and none have provided a comprehensive analysis of different Cbl species.

Methylation of DNA and histone proteins complexly regulates gene expression and this form of epigenetic regulation is particularly important during development, including pre- and postnatal brain development (Numata S, Ye T, Hyde T M, Guitart-Navarro X, Tao R, Wininger M. et al. DNA methylation signatures in development and aging of the human prefrontal cortex. Am J Hum Genet. 2012; 90:260-272). Neural tube defects, as well as Rett and Angelman/Prader-Willi neurodevelopmental syndromes are linked to defects in methylation-dependent epigenetic regulation (Gapp K, Woldemichael B T, Bohacek J, Mansuy I M. Epigenetic regulation in neurodevelopment and neurodegenerative diseases. Neuroscience. 2014; 264:99-111; Imbard A, Benoist J F, Blom H J. Neural tube defects, folic acid and methylation. Int J Environ Res Public Health. 2013; 10:4352-43589; and Guy J, Cheval H, Selfridge J, Bird A. The role of MeCP2 in the brain. Annu Rev Cell Dev Biol. 2011; 27:631-652). Turnover of DNA methylation marks is very fast in prefrontal cortex during fetal development but is 2-3 orders lower during childhood and later life (Numata S et al. DNA methylation signatures in development and aging of the human prefrontal cortex. Am J Hum Genet. 2012, 90: 260-272. Erratum in: Am J Hum Genet. 2012 Oct 5; 91: 765). It has been shown that the level of MS mRNA in human prefrontal cortex decreases several hundred-fold across the lifespan, indicating a dynamic role for vitamin B₁₂-dependent MS activity in brain development and function, and MS mRNA levels were prematurely decreased in autistic subjects (Muratore CR et al. Age-dependent decrease and alternative splicing of methionine synthase mRNA in human cerebral cortex and an accelerated decrease in autism. PloS One. 2013; 8: e56927). Abnormal DNA methylation (Ladd-Acosta C, Hansen K D, Briem E, Fallin M D, Kaufmann W E, Feinberg A P. Common DNA methylation alterations in multiple brain regions in autism. Mol Psychiatry. 2014; 19:862-871 and James S J, Shpyleva S, Melnyk S, Pavliv O, Pogribny I P. Elevated 5-hydroxymethylcytosine in the Engrailed-2 (EN-2) promoter is associated with increased gene expression and decreased MeCP2 binding in autism cerebellum. Transl Psychiatry. 2014; 4: e460) has been reported in postmortem brain of autistic subjects, in conjunction with low levels of the antioxidant GSH and elevated markers of oxidative stress (Rose S, Melnyk S, Pavliv O, Bai S, Nick T G, Frye R E. et al. Evidence of oxidative damage and inflammation associated with low glutathione redox status in the autism brain. Transl Psychiatry. 2012; 2: e134 and Chauhan A, Audhya T, Chauhan V. Brain region-specific glutathione redox imbalance in autism. Neurochem Res. 2012; 37:1681-1689). Increased oxidative stress and impaired methylation have also been implicated in schizophrenia (Prabakaran S, Swatton J E, Ryan M M, Huffaker S J, Huang J T, Griffin J L. et al. Mitochondrial dysfunction in schizophrenia: evidence for compromised brain metabolism and oxidative stress. Mol Psychiatry. 2004; 9:684-697 and Do K Q, Cabungcal J H, Frank A, Steullet P, Cuenod M. Redox dysregulation, neurodevelopment and schizophrenia. Curr Opin Neurobiol. 2009; 19:220-230).

Zhang et al. (Decreased Brain Levels of Vitamin B12 in Aging, Autism and Schizophrenia. PLoS One. 2016; 1-19) utilized a HPLC/electrochemical detection-based assay to quantify individual Cbl species in postmortem human cerebral cortex of control subjects from fetal to 80 yrs of age, as well as autistic and schizophrenic subjects. Changes in Cbl species were compared with the status of methylation and antioxidant pathway metabolites and the influence of decreased GSH production on brain Cbl levels was evaluated in glutamate-cysteine ligase modulatory subunit knockout (GCLM-KO) mice in which GSH synthesis was impaired, leading to a brain GSH level decrease of 60-70% (Steullet P, Cabungcal J H, Kulak A, Kraftsik R, Chen Y, Dalton T P. et al. Chronic Redox Dysregulation Affects the Ventral But Not Dorsal Hippocampus: Impairment of Parvalbumin Neurons, Gamma Oscillations and Related Behaviors. J Neurosci. 2010; 30:2547-2558). The results revealed an unexpected decrease in cortical Cbl and MeCbl levels across the lifespan, as well as premature decreases in both autism and schizophrenia, which were replicated in GCLM-KO mice.

Taken together, the above-described data provides a strong rationale for pursuing therapeutic and/or nutritional interventions aimed at augmenting methylation capacity in the brain.

### SUMMARY OF THE INVENTION

The invention provides methods and cobalamin (vitamin B₁₂) nutraceutical compositions for improving cognitive function in subjects suffering from disorders of cognitive function such as neurodegenerative disorders, neurodevelopmental disorders, and neuropsychiatric disorders.

This improvement in cognitive function is achieved through the ability of the cobalamin (vitamin B₁₂) nutraceutical compositions to increase methylation capacity and vitamin B₁₂ activity the brain of the subjects.

### DETAILED DESCRIPTION OF THE INVENTION

For the purpose of promoting an understanding of the principles of the invention, reference will now be made to embodiments illustrated herein and specific language will be used to describe the same. It will nevertheless be understood that no limitation of the scope of the invention is thereby intended. Any alterations and further modification in the described compositions, pharmaceutical/nutraceutical formulations, methods, therapeutic treatments, and any further application of the principles of the invention as described herein, are contemplated as would normally occur to one skilled in the art to which the invention relates.

Methylation reactions regulate almost every aspect of metabolism, including epigenetic regulation of gene expression. Methylation is controlled by the vitamin B₁₂ and folate-dependent enzyme methionine synthase (MS), through its dual influence over the methyl donor S-adenosylmethionine (SAM) and the methylation inhibitor S-adenosylhomocysteine (SAH). It has been shown, in a postmortem human brain study, that MS mRNA levels in frontal cortex decrease dramatically across the lifespan (˜400-fold), accompanied by increased alternative splicing (Muratore CR et al. Age-dependent decrease and alternative splicing of methionine synthase mRNA in human cerebral cortex and an accelerated decrease in autism. PloS One. 2013; 8: e56927), indicating that methylation is dynamically regulated in the brain. Recently, it has been reported that brain levels of vitamin B₁₂ levels decrease with age, with the level of methyl B₁₂ being >10-fold lower in 60-80 yr old control subjects as compared to 0-20 yr old subjects (Zhang et al. Decreased Brain Levels of Vitamin B12 in Aging, Autism and Schizophrenia. PLoS One. 2016; 1-19). This highly significant decrease is not mirrored in plasma levels, indicating that deficits in brain B₁₂ are not detected by routine B₁₂ testing. Additionally, 3-fold lower brain B₁₂ levels in autistic and schizophrenia subjects has been found, suggesting that low B₁₂ may be a feature of brain disorders.

A large number of studies implicate impaired methylation status in Alzheimer's disease (AD), including elevated plasma and brain levels of homocysteine, the substrate of MS, and decreased levels of SAM. Hyperhomocysteinemia and DNA hypomethylation are associated with elevated presenillinl expression and Abeta (amyloid beta) production. Impaired SAM-dependent methylation of Tau protein and the Tau phosphatase PP2A can contribute to neurofibrillary tangle formation. The VITACOG study, conducted in the UK, showed significant clinical benefit of treatment with vitamin B₁₂, folic acid and vitamin B₆ in mild cognitive impairment (MCI), including diminished gray matter loss. Importantly, follow-up analyses showed that a therapeutic response was restricted to individuals in the two higher percentiles of plasma omega-3 fatty acids (primarily DHA), suggesting a crucial role for methylation of DHA-containing membrane phospholipids. The instant inventors were the first to describe the unique ability of the D4 dopamine receptor to carry out MS-dependent phospholipid methylation in human neuronal cells in response to dopamine and proposed its role in attention (Sharma A, Kramer M L, Wick P F, Liu D, Chari S, Shim S. et al. D4 dopamine receptor-mediated phospholipid methylation and its implications for mental illnesses such as schizophrenia. Mol Psychiatry. 1999; 4:235-246). Subsequent research confirmed D4 receptor involvement in gamma synchrony during attention (Kocsis, B, Lee, P, Deth, R. Brain Struct Funct 2014 219(6) 2173-2180). The specific phospholipid that is primarily methylated by the D4 receptor contains DHA. Recent and as yet unpublished results in cultured cells show that D4 receptor activation by dopamine is associated with increased antioxidant (glutathione or GSH) levels, a 3-fold increase in SAM/SAH and a 2-fold increase of global DNA methylation.

Oxidative stress is a well-recognized risk factor for AD and other neurodegenerative diseases. It has been shown that insulin-like growth factor 1 (IGF1) stimulates cysteine uptake in neuronal cells, leading to increased GSH levels and improved methylation status (Hodgson N, Trivedi M, Muratore C, Li S, Deth R. Soluble oligomers of amyloid-β cause changes in redox state, DNA methylation, and gene transcription by inhibiting EAAT3 mediated cysteine uptake. J Alzheimers Dis. 2013; 36:197-209.). Moreover, Amyloid Beta had the opposite effect, causing a decrease in cysteine uptake and GSH, while antagonizing the effect of IGF1.

Furthermore, it has been shown that treatment of patients with Alzheimer's disease, who had elevated homocysteine level, with a B₁₂-based nutraceutical, CerefolinNAC® (commercial name), caused a highly significant decrease in brain atrophy. CerefolinNAC® contains L-methylfolate, methylcobalamin, and N-acetyl-cysteine. (Shankle, W R et al. CerefolinNAC® Therapy of Hyperhomocysteinemia Delays Cortical and White Matter Atropy in Alzheimer's Disease and Cerebrovascular Disease, Journal of Alzheimer's Disease, 2016 54:1073-1084). N-acetyl-cysteine promotes formation of the antioxidant glutathione, but a combination of methylcobalamin and hydroxocobalamin leads to increased glutathione in the absence of N-acetyl-cysteine. The above-described experimental results provide a strong rationale for pursuing formulations designed to optimize vitamin B₁₂ activity in the brain and/or formulations for augmenting methylation capacity in the brain.

### KEY SUPPORTIVE DATA POINTS

Alzheimer's disease (AD) and other neurodegenerative disorders are associated with oxidative stress.

Oxidative stress inhibits folate and vitamin B₁₂-dependent methionine synthase (MS), resulting in inhibition of ˜1,000 methylation reactions, including DNA and histone methylation.

Impaired MS activity and impaired methylation are features of AD.

The recent VITCOG clinical trial clearly demonstrated benefit of a B₆, B₁₂ and folate supplement in mild cognitive impairment (MCI), including decreased gray matter loss and cognitive improvement.

Analysis of responders vs. in the VITACOG trial showed that only individuals in the upper two percentiles of DHA (omega-3 fatty acid) benefitted from the treatment.

Recent postmortem brain study showed an age-dependent decline in brain levels of vitamin B₁₂, particularly the methylB₁₂ form, which was 10-fold lower in 60-80 yr old subjects vs. 0-20 yr old subjects.

The incidence of neurodegenerative disorders is low in bipolar subjects because of their treatment with lithium.

Lithium affects the transport process that bring vitamin B₁₂ into the brain.

The instant inventors have developed a sublingually-deliverable nutraceutical composition designed for improving cognitive functions. The composition contains at least methylcobalamin and hydroxycobalamin, separately or as combined vitamin B12. In addition to methylcobalamin and hydroxycobalamin, the composition can also contain methylfolate or methylfolate and lithium. The inclusion of lithium includes “low dose” forms.

In one aspect, the invention provides a pharmaceutical or nutraceutical composition including therapeutically-effective amounts of methylcobalamin, hydroxycobalamin, methylfolate, and lithium and at least one pharmaceutically-acceptable carrier or non-medicinal ingredient (NMI).

In another aspect, the invention provides a pharmaceutical or nutraceutical composition including therapeutically-effective amounts of methylcobalamin, hydroxycobalamin, methylfolate, and at least one pharmaceutically-acceptable carrier or non-medicinal ingredient (NMI).

In yet another aspect, the invention provides a pharmaceutical or nutraceutical composition including therapeutically-effective amounts of methylcobalamin, hydroxycobalamin, and at least one pharmaceutically-acceptable carrier or non-medicinal ingredient (NMI).

As used herein, the phrase “improving cognitive functions” refers to bringing mental processes into a more desired or better functioning condition.

As used herein, a “nutraceutical” refers to a pharmaceutical grade nutrient or food ingredient.

The composition is formulated for sublingual delivery to a subject. Sublingual delivery differs from conventional oral delivery in that the tablet disintegrates in the mouth of the subject.

The term “subject” includes any human being or animal who would benefit from improved cognitive function; i.e. having methylation capacity of the brain increased and/or vitamin B₁₂ activity in the brain optimized. The terms “patient” and “human patient” are also used herein to refer to the subject.

The phrase “optimize vitamin B₁₂ activity” refers to one or both of an increase in amount of activity and an increase in effectiveness of the activity. The vitamin B₁₂ activity is preferentially increased within the brain, but is not limited to an increase therein.

In one embodiment, the carotenoid antioxidant, Astaxanthin, can be included within the formulation. Ingredients, in the suggested ranges, for this formulation include: Methylcobalamin—from about 1 to about 10 mg; Hydroxocobalamin—from 0 to about 5 mg; Astaxanthin-from about 1 to about 10 mg; and Lithium-from about 0.1 to about 1 mg. One particular embodiment of the formulation of the composition includes: about 2 mg of combined vitamin B12 (as methylcobalamin about 1600 mg; hydroxycobalamin about 400 mg); about 400 mcg methylfolate; and about 5 mg of lithium (equivalent to about 130 mg lithium orotate).

Another embodiment of the formulation of the composition includes: about 2 mg of combined vitamin B12 (as methylcobalamin about 1600 mg; hydroxycobalamin about 400 mg) and about 400 mcg methylfolate.

Another embodiment of the formulation of the composition includes: about 2 mg of combined vitamin B12 (as methylcobalamin about 1600 mg; hydroxycobalamin about 400 mg).

In describing the formulations, the term “about” refers to near or close to the disclosed quantities and encompasses quantities in which the composition can be formulated with and still reasonably achieve the described and/or desired function of the formulation. Additionally, when referring to amounts of the pharmaceutical composition, the term “about” refers to dosages at or near the stated amounts at which the desired function can be achieved.

The phrase “pharmaceutically-acceptable carrier” refers to an inactive and non-toxic substance used in association with an active substance, i.e. in this formulation the active substances are preferably, but not limited to, methylcobalamin, hydroxocobalamin, methylfolate, and lithium, especially for aiding in the application/delivery of the active substance. Non-limiting examples of pharmaceutically-acceptable carriers are diluents, binders, disintegrants, superdisintegrants, flavorings, fillers, and lubricants. Pharmaceutically-acceptable carriers can have more than one function within a formulation, a non-limiting e.g. a filler can also be a disintegrant. Additionally, pharmaceutically-acceptable carriers may also be referred to as non-medicinal ingredients (NMIs) or pharmaceutically-acceptable excipients.

The phrase “effective amount” and refers to the amount of a composition/formulation necessary to achieve the intended function of the composition/formulation. An “effective amount” can also be referred to as a “therapeutically-effective amount.”

The described pharmaceutical/nutraceutical compositions have various applications/functions. Non-limiting examples include increasing methylation capacity in the brain, elevating levels of methylcobalamin in the brain, increasing plasma cobalamin and antioxidant levels, increasing cellular cobalamin and antioxidant levels, optimizing vitamin B12 activity in the brain, increasing energy and alertness, and improving cognitive functions. Glutathione (GSH) is a non-limiting example of an antioxidant of which levels may be increased.

In one aspect, the invention provides a method for increasing methylation capacity in a brain of a subject in need thereof or who could benefit therefrom. The method includes steps for providing a cobalamin composition described herein and administering the cobalamin to the subject to achieve increased methylation capacity in the brain of the subject.

In another aspect, the invention provides a method for elevating levels of methylcobalamin in a brain of a subject in need thereof or who could benefit therefrom. The method includes steps for providing a cobalamin composition described herein and administering the cobalamin to the subject to achieve elevated levels of methylcobalamin in the brain of the subject.

In yet another aspect, the invention provides a method for increasing cobalamin (plasma and/or cellular) and antioxidant levels (such as, but not limited to, glutathione) in a subject in need thereof or who could benefit therefrom. The method includes steps for providing a cobalamin composition described herein and administering the cobalamin to the subject to achieve increased cobalamin (plasma and/or cellular) and antioxidant levels in the subject.

Any of the pharmaceutical/nutraceutical cobalamin compositions described herein, which have been formulated for sublingual administration, are contemplated for use with the methods descried herein.

In another aspect, any of the above-described epinephrine fine particles (including epinephrine nanoparticles or nanocrystals and epinephrine microparticles or microcrystals) can be used in the manufacture of any of the above-described compositions and pharmaceutical compositions.

In another aspect, any of the above-described ingredients, methylcobalamin, hydroxycobalamin, methylfolate, and lithium, can be used in the manufacture of any of the above-described pharmaceutical/nutraceutical compositions.

The novel cobalamin compositions described herein are contemplated for therapeutic use in conditions which can benefit from improved cognitive function and/or optimized vitamin B₁₂ activity (in the brain); such as, but not limited to, neurodegenerative disorders, neurodevelopmental disorders, and neuropsychiatric disorders. Specific, but non-limiting examples of such disorders are Alzheimer's disease (AD), mild cognitive impairment (MCI), chronic fatigue syndrome/myalgic encephalomyelitis (CFS/ME), aging, autism, schizophrenia, and attention deficit hyperactivity disorder (ADHD).

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not intended to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The compositions, nutraceutical formulations, pharmaceutical/therapeutic compositions and methods, procedures, and techniques described herein are presently representative of the preferred embodiments, are intended to be exemplary and are not intended as limitations on the scope. Changes therein and other uses will occur to those skilled in the art which are encompassed within the spirit of the invention. Although the invention has been described in connection with specific, preferred embodiments, it should be understood that the invention as ultimately claimed should not be unduly limited to such specific embodiments. Indeed various modifications of the described modes for carrying out the invention which are obvious to those skilled in the art are intended to be within the scope of the invention.

1-42. (canceled)

43. A nutraceutical composition comprising methylcobalamin, hydroxycobalamin, and methylfolate.

44. The nutraceutical composition according to claim 43, wherein the methylcobalamin and hydroxycobalamin are combined as vitamin B₁₂.

45. The nutraceutical composition according to claim 44, further comprising lithium.

46. The nutraceutical composition according to claim 45, wherein the lithium is lithium orotate.

47. A pharmaceutical composition for increasing methylation capacity in a brain of a subject, the pharmaceutical composition comprising a therapeutically-effective amount of the nutraceutical composition according to claim 43 and at least one pharmaceutically-effective carrier or non-medicinal ingredient.

48. The pharmaceutical composition according to claim 47, wherein the methylcobalamin and hydroxycobalamin are combined as vitamin B₁₂.

49. The pharmaceutical composition according to claim 48, further comprising lithium.

50. The pharmaceutical composition according to claim 49, wherein the pharmaceutical composition is formulated for sublingual administration.

51. A pharmaceutical composition for increasing methylation capacity in a brain of a subject, the pharmaceutical composition comprising therapeutically-effective amounts of methylcobalamin, hydroxycobalamin, methylfolate, and lithium and at least one pharmaceutically-acceptable carrier or non-medicinal ingredient (NMI).

52. The pharmaceutical composition according to claim 51, wherein the pharmaceutical composition is formulated for sublingual administration.

53. The pharmaceutical composition according to claim 52, wherein the methylcobalamin and hydroxycobalamin are combined as vitamin B₁₂.

54. The pharmaceutical composition according to claim 53, wherein the lithium is lithium orotate.

55. The pharmaceutical composition according to claim 54, wherein the therapeutically-effective amount of methylcobalamin and hydroxycobalamin combined as vitamin B₁₂ is about 2 mg, the therapeutically-effective amount of methylfolate is about 400 mcg, and the therapeutically-effective amount of lithium orotate is 130 mg.

56. The pharmaceutical composition according to claim 55, further comprising a therapeutically-effective amount of astaxanthin.

57. A method for increasing methylation capacity in a brain of a subject in need thereof, the method comprising:
providing a pharmaceutical composition formulated for sublingual administration comprising therapeutically-effective amounts of methylcobalamin, hydroxycobalamin, methylfolate, and lithium and at least one pharmaceutically-acceptable carrier or non-medicinal ingredient (NMI); and
administering the pharmaceutical composition to the subject, thereby increasing methylation capacity in the brain of the subject.

58. A method for elevating levels of methylcobalamin in a brain of a subject in need thereof, the method comprising:
providing a composition formulated for sublingual administration comprising the pharmaceutical composition according to claim 51; and
administering the composition to the subject, thereby elevating levels of methylcobalamin in the brain of the subject.

59. A method for increasing plasma cobalamin and antioxidant levels in a subject, the method comprising:
providing a composition formulated for sublingual administration comprising the pharmaceutical composition according to claim 51; and
administering the composition to the subject, thereby increasing plasma cobalamin and antioxidant levels in the subject.

60. The method according to claim 59, wherein increasing antioxidant levels includes increasing glutathione (GSH) levels in the subject.

61. A method for increasing cellular cobalamin and antioxidant levels in a subject, the method comprising:
providing a composition formulated for sublingual administration comprising the pharmaceutical composition according to claim 51; and
administering the composition to the subject, thereby increasing cellular cobalamin and antioxidant levels in the subject.

62. The method according to claim 61, wherein increasing antioxidant levels includes increasing glutathione (GSH) levels in the subject.
